(19) United States
(12) Patent Application Publication
(10) Pub. No.: US 2026/0354399 A1
(43) Pub. Date: Nov. 12, 2026

(54) THAWING DEVICE FOR SAMPLING OF FROZEN PRODUCTS

(71) Applicant: Fremonta Corporation, San Jose, CA (US)

(72) Inventors: Eric WILHELMSEN, San Jose, CA (US); Yongqing HUANG, San Jose, CA (US); Yicun HUANG, San Jose, CA (US)

(21) Appl. No.: 19/488,056

(22) PCT Filed: May 13, 2024
(86) PCT No.: PCT/US2024/029115

§ 371 Date: Nov. 25, 2025

Related U.S. Application Data
(60) Provisional application No. 63/501,974, filed on May 12, 2023.

(51) Int. Cl. A23B 4/07; A23B 2/82; G01N 1/04; G01N 1/44; G01N 33/02

(52) U.S. Cl. CPC A23B 4/07; A23B 2/82; G01N 1/04; G01N 1/44; G01N 33/02

(57) ABSTRACT

Devices, system and methods for sampling frozen food products are provided. Devices can include thawing devices having a cover forming an enclosure over containers of frozen food products and an air heating unit mounted in the cover and having a airflow output disposed in an opening in the cover to direct heated air across the frozen product sufficiently to thaw a surface of the food product for sampling. The heated air unit can include controls for controlling a heating level and/or duration of heating to thaw the surface. Sampling systems can include the thawing device and a sampling sheet or mitt for directly contacting the thawed surface to obtain an aggregated sample. Methods include exposing frozen food in a container to heated air via the thawing device and subsequently contact sampling the thawed surface with a sampling sheet or mitt and testing the aggregated sample for target analytes.

### CROSS-REFERENCES TO RELATED APPLICATIONS

This application claims the benefit of priority of U.S. Provisional Application No. 63/501,974 filed May 12, 2023, which is incorporated herein by reference in its entirety.

### FIELD OF THE INVENTION

The present invention relates to sampling of frozen products, in particular devices and methods for thawing frozen products for obtaining microbial surface sampling.

### BACKGROUND

Many perishable products are stored and shipped frozen. Many of the products are subject to testing. This testing can be used to verify conformity to specifications and/or regulations. In some cases, the testing is part of a safety program such as when pathogen screening is done on frozen meat.

In commercial practice, this sampling may be done by mechanically extracting a portion of the material. This is commonly done with an electric drill with the goal of accumulating 150 g of shavings. Alternatively, the material is thawed which takes time and space. It is taught that in many cases, it is not necessary to thaw the entire lot or portion for sampling if a surface sample will suffice, such as described in U.S. application Ser. No. 17/898,178 While these methods represent a marked advancement in frozen food sampling, the thawing and sampling process tends to be time-consuming and laborious for the sampler.

Thus, there is a need for improved methods and devices for sampling of frozen or solid products that overcome the challenges noted above.

### BRIEF SUMMARY OF THE INVENTION

In one aspect, the present invention is directed to devices and method for improving sampling of frozen products, particularly devices and methods that allow scaling up frozen sampling to allow lot clearance of a lot of food products, such as frozen beef trim. A thawing device is provided that reduces labor and facilitates lot clearance. In another aspect, the device and methods herein provide a non-contact approach to thawing the surface which greatly increases the tolerance for an uneven sampling surface, as compared to conventional approaches.

In a first aspect, the invention pertains to a thawing device to facilitate sampling of frozen food products. The thawing device can include: a cover sized to fit over an opening of a container that contains frozen food products such that when the cover is fit over the container the cover forms an enclosure; and an air heating unit mounted in the cover, the air heating unit having a heated airflow output disposed in an opening in the top of the cover to facilitate heated air flow into the enclosure. Such thawing devices can further include one or more controls operatively coupled to the air heating unit, the one or more controls being configured to control a heating level and/or duration of operation of the air heating unit so as to sufficiently thaw the surface of the frozen food for sampling without completely thawing the frozen food product. In some embodiments, the cover includes one or more openings to allow air within the enclosure to escape so that heated air flows into the enclosure from the air heating unit. In some embodiments, the one or more controls include a heating level control configured to select from multiple heating levels or fan only setting. In some embodiments, the one or more controls further include a timer control configured to select the duration of time of operation of the air heated. In some embodiments, the cover further includes one or more coupling features, such as a latch mechanism, to secure the cover atop the container. The cover can be shaped to correspond to the container having the frozen product, typically rectangular or square in shape. In some embodiments, the cover is about 12×12 inches about a perimeter of the bottom opening. The cover can further have a height between 6 and 12 inches so as to form the enclosure and to space the air flow output a suitable distance away from the frozen food product.

In another aspect, the invention pertains to a sampling system for sampling frozen foods. The sampling system can include the thawing device, as detailed above, and an aggregate sampler. The aggregate sampler can include a sampling sheet or mitt for sampling by direct contact with the thawed surface. In some embodiments, the sampling mitt includes one or more sheets of absorbent sampling medium, typically a non-woven polypropylene fabric, having seams to form a pocket for a user's hand to facilitate manual sampling. The sampling mitt can be any suitable size or shape (e.g. 5×5 inches, 8×8 inches, 8×12 inches, etc.).

In yet another aspect, the invention pertains to a method of sampling a frozen product. The method can include steps of: placing a thawing device atop a container of frozen food product, the device having an air heating unit disposed in a cover forming an enclosure over the container; operating the air heating unit to direct heated air into the enclosure and over the frozen food product to thaw the surface; and sampling the thawed surface of the frozen food product for analytical testing. The method can further include subsequently testing the aggregated sample for a target analyte to clear the frozen food product or clear an entire lot associated with the frozen food product. Aggregate sampling can include directly contacting the thawed surface with a sampling medium to obtain an aggregated sample. In some embodiments, the sampling member is a sampling sheet or sampling mitt that is manually rubbed across the thawed surface. In some embodiments, the method further includes selecting a heat level based on a type and/or temperature of the frozen food product, and/or selecting a duration of operation of the air heating unit of the frozen food product. After a sufficient duration of time to thaw the surface, operation of the heating unit can be ceased, either automatically or manually by the user, the cover removed and the surface aggregate sampled by the user for testing.

The method can further include placing the thawing device atop another container of frozen food product forming an enclosure over the container; operating the air heating unit to direct heated air into the enclosure and over the frozen food product to thaw the surface; and sampling the thawed surface of the frozen food product for analytical testing. This method can be repeated with multiple units of frozen food product, using the same aggregate sampler for all units or using a new aggregate sampler each time.

### BRIEF DESCRIPTION OF THE DRAWINGS

FIG. 1 shows a thawing device to facilitate controlled thawing of frozen food product for biological surface sampling, in accordance with some embodiments.

FIGS. 2A-2D show various views of the thawing device of FIG. 1, in accordance with some embodiments.

FIG. 3 shows a heating unit of the thawing device, in accordance with some embodiments.

FIGS. 4-9 show steps of sampling frozen food products using a thawing device, in accordance with some embodiments.

FIG. 10 shows an exemplary sampling sheet for microbial samplings, which can be used in sampling frozen food products using a thawing device, in accordance with some embodiments.

FIGS. 11-12 depict methods of sampling frozen food products, which can utilize a thawing device in accordance with some embodiments.

FIGS. 13-14 show alternative thawing devices to facilitate controlled thawing of frozen food product for biological surface sampling, in accordance with some embodiments.

### DETAILED DESCRIPTION OF THE INVENTION

The present invention pertains to improved sampling devices and methods for sampling of frozen food product, particularly beef trimmings. In order to facilitate microbial sampling of frozen food products, it is advantageous to at least partly melt a surface of the frozen food product in order to obtain a surface sample that avoids the need to entirely thaw the entire food product, as described in U.S. application Ser. No. 17/898,178, which details various ways to deliver energy to this surface to effected the melting and avoiding undesirable changes in the sampled material. However, there are difficulties and challenges associated with these conventional approaches, including uneven surfaces and the considerable time and effort in manipulating heated samplers on frozen food products to thaw the surface. Presented herein are devices and methods that improve upon conventional approaches to facilitate sampling lots of frozen materials, particularly food products. The improved devices and method overcomes the challenges of uneven surfaces and reduces the effort involved on the part of the sampler when sampling multiple units of product as usually required for lot clearance.

In one aspect, the thawing device uses heated air in a controlled environment in order to provide improve and uniform thawing of a surface of the frozen product, typically frozen beef or poultry, to facilitate surface sampling. In some embodiments, the thawing device uses a heating unit that provides controlled flow of heated air to thaw a surface of the frozen product to be sampled. In some embodiments, the heating unit uses ohmic heating to heat air that is passed over the surface to be melted. In some embodiments, the heating unit further includes a fan provides the air movement and turbulence to melt the surface layer of the product. Advantageously, the turbulence in the air provides hot air contact with the entire surface regardless of unevenness. Accordingly, the surface is reliably thawed sufficiently for sampling with minimal effort from the user.

In some embodiments, the thawing device uses an enclosure that fully or partially encloses the frozen product so that the heated air can be more readily controlled and directed to the frozen product. It is useful to mostly confine the air to a small volume over the surface to be sampled to allow efficient heat exchange. In some embodiments, this confinement is not complete to allow air to flow out of the small volume. For example, the enclosure cover can include gaps and/or holes to allow air flow out of the enclosure to be replaced by heated air blown by the heating unit into the enclosure.

In some embodiments, the thawing device can include a heating control to allow a user to control the temperature. In some embodiments, the thawing device can include a temperature sensor within the enclosure and a thermostat so as to automatically adjust the heated air as needed to maintain an optimal thawing temperature without damaging the product or partially cooking the frozen food product.

In some embodiments, the thawing device includes a timer or clock to control or monitor a duration of time of application of heated air (e.g. 2-8 minutes, 2-5 minutes, 2-4 minutes, about 3 minutes) to ensure thawing of the surface for sampling. In some embodiments, the timer can be set to control heating after which the heating unit automatically turns off after the pre-set duration of time. In other embodiments, the device can include a clock indicating the elapsed time for monitoring by the user.

Melting times between 2 and 5 minutes have proven useful with about 3 minutes representing a good balance between time and melting. It is appreciated that the melting time will be a function of the sample temperature, the wattage of the heating element and the captured volume. For example, products held at ultra-cold storage may require additional time (e.g. 2-8 minutes) to sufficiently thaw for microbial surface sampling. In any case, it is desirable to avoid overheating the sample, which can cause damage or partial cooking of the product. When multiple units are to be sampled, successive units can be melted while the previous unit is being sampled. This approach provides for better time management for the sampler.

In some embodiments, the device includes a heating unit that is integrated with a cover of the enclosure. The heating unit can include a heated air outflow (e.g. vent) disposed within the enclosure cover. The cover can include gaps, holes or other such openings, typically near the bottom, to allow cold air within the enclosure to escape while heated air is directed into the enclosure. The cover is sized to fit atop a container or tray on which the product is placed. The cover can further includes one or more latches for securing the cover on the container. The container may also be a box or carton and may come in various differing sizes, such as shown below in Table 1. While these sizes are typical for boxed beef, it is appreciated that these and various other sizes could be used for various other types of frozen food, or other frozen products.

TABLE 1
Frozen Food Carton Sizes
Frozen Boxed Beef Dimensions (Inches)

| W | L | H |
|---|---|---|
| 13.50 | 20.75 | 6.50 |
| 13.50 | 20.50 | 6.00 |
| 13.75 | 20.50 | 6.50 |
| 13.50 | 20.75 | 6.25 |
| 14.50 | 21.75 | 6.00 |
| 13.75 | 21.50 | 6.50 |
| 15.50 | 22.50 | 6.00 |
| 16.00 | 23.00 | 8.00 |

Once the surface of the product is sufficiently thawed by the device, the cover can be removed for microbial sampling of the product surface. In some embodiments, the device can provide an alert (e.g. audible beep, light) to indicate that sufficient thawing time has elapsed so that the user can proceed with sampling. An alert can be useful since once the heating unit is turned off, the remaining thermal mass of frozen product can quickly refreeze the surface.

An aggregated sampling device can be used for collecting the melted material from the surface after heating. Sampling can be performed with any suitable sampling medium, such as an absorbent cloth or sheet that adsorbs or absorbs particles, residue and/or liquid from the thawed surface of the product being sampled. However, alternative approaches for collecting the melt could be realized. An absorbent sampling medium is preferred for ease of use and consistency in sampling. In some embodiments, non-woven polyolefin fabric is particularly useful because it leaves no residues and is easily sterilized. The fabric cloth can be provided in the form of a simple sheet or swab. It can be also be configured favorably into a pouch or mitt to facilitate rubbing over the thawed surface. The MicroTally® Sheet or Mitt offered by FREMONTA Corp is especially effective when each side is sampled for 45 seconds on each side of the sampling sheet or mitt for each unit. However, shorter and longer times are useful in some sampling situations. It is appreciated that any suitable sampling medium can be used and different sampling protocols can be applied. After sampling, the sampling sheet or mitt can be folded and placed back in the bag for subsequent testing.

Preferably, the device can be configured so that no part of the beating unit contacts the sample directly, effectively eliminating concerns about cross contamination. This distinction affords the opportunity for including the basic heating mechanism into more complete sample handling systems. Such an approach may increase cost and complexity of the device, but may increase user safety and ease of use and reduce contamination between lots of product being sampled. Advantageously, the thawing device could potentially be used on multiple units of frozen products without cross-contamination. Some of these potential improvements are discussed as part of the beef trim example below. It is noted that the enclosure cover that attaches to the heating unit can also be readily removable to allow for ease of washing and decontamination, as needed.

After surface sampling is performed, the aggregated sample can be analyzed for a wide range of analytes. The general process is to extract the absorbed material, perform any required sample preparation and apply a detection methodology. Testing using enrichment procedures or testing that detects target analytes without any enrichment can be used. Aspects of aggregate sampling can be further understood by referring to the aggregating sampling devices and operations described in U.S. application Ser. Nos. 16/057,137; 16/525,350; and 63/134,671, the entire contents of which are incorporated herein by reference for all purposes. Testing for bacterial pathogens is of particular interest and can optionally include additional aspects and specifics of pathogen sampling and testing, such as any of those described in U.S. application Ser. No. 17/956,308 [Atty Docket No. 102974-000610US].

An illustrative example of sampling frozen beef trim is detailed below. First, frozen beef trim product is provided in a plastic tray or box having a top opening of a known size (e.g. 12.5″L×12.5″W). A thawing device having an air heating unit (e.g. electric heated fan mounted) in a correspondingly sized hole in the top of a plastic cover (e.g. 12.5″L×12.5″W×7″H) is placed over the tray, the cover forming an enclosure over the frozen beef trim. In some embodiments, the heated fan has one or more settings (e.g. high/low, wattage settings). It is appreciated that the cover should be size to correspond to the top opening of the container or tray holding the product. In the above example, the cover was sized for the particular tray or box opening shown. It is appreciated that the cover can be sized according to various other sizes as well. Other sizes and arrangements to adjust the air confinement area can further increase the utility of the device.

In some embodiments, the heated fan has at least two wattage settings (e.g. 750 W and 1500 W). Small rectangular holes in the sides of the cover allow air flow through the enclosure. The placement of the heated fan in the top of the cover allows the air flow to be directed over the surface to be melted, while contact of the heated fan and the food product is avoided. The heated fan is operated for a sufficient time to thaw the surface of the frozen food product for sampling. Various times were explored but about three minutes was found to be adequate. Longer times provided no apparent sampling advantage and risked damaging/cooking the food product. However, it is important to note the sampling needs to be done very soon or immediately after the heating is completed since the bulk of the frozen material rapidly refreezes the thin melted layer which limits sampling effectiveness.

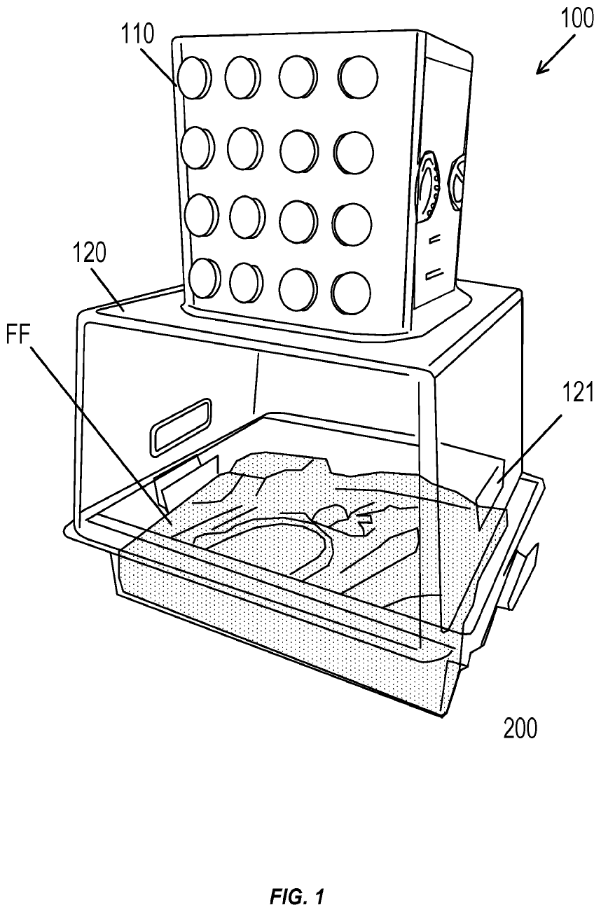

Additional details of this implementation can understood by referring to FIGS. 1-10. It is appreciated that these concepts are not limited to this implementation and that various other embodiments can be realized, for example scaled up versions for larger containers.

FIG. 1 shows an exemplary thawing device 100 to facilitate controlled partial thawing of frozen product for biological surface sampling, in accordance with some embodiments. As described above, the frozen product can be frozen food products, such as beef trim, poultry or fish. The thawing device 100 includes a heating unit 110, which is mounted in a top opening in a cover 120 that fits atop container 200 filled with frozen food FF, such as frozen beef trim. When placed atop the container 200, the cover forms an enclosure over the frozen food FF so as to allow improved control of the micro-environment of the frozen food to facilitate rapid thawing. In this embodiment, the cover 120 includes holes 121 on each side, which allow outflow of ambient air inside the enclosure as heated air is blown into the enclosure from the heater unit 110. While the container 200 here is shown as a tray specially adapted to fit and lock with the cover 120, it is appreciated that container could be any suitable container, including a box, that contains frozen food and is not necessarily require to be specially fitted or securely attachable to the cover. It is desirable however, for the cover to be sized according to the top opening of the container to provide a partially contained environment inside the enclosure.

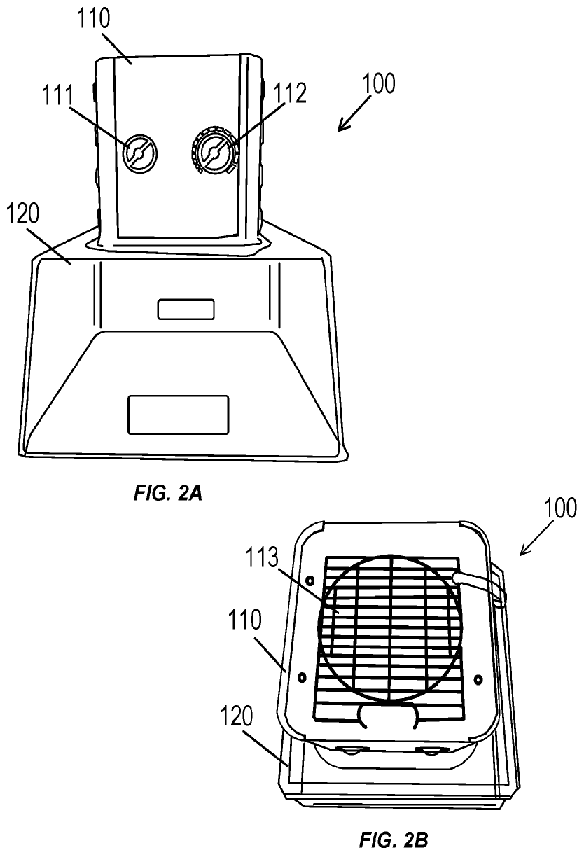

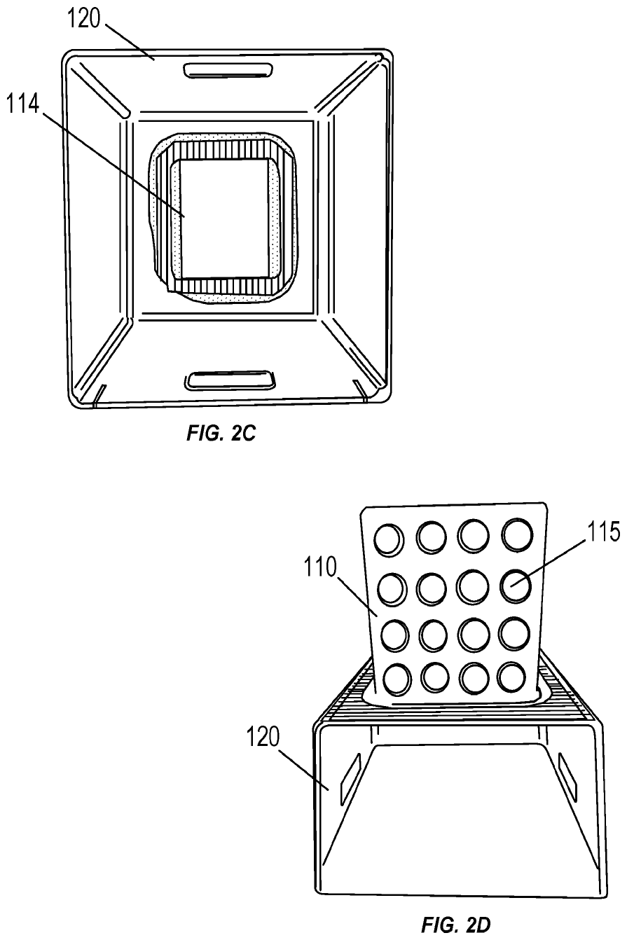

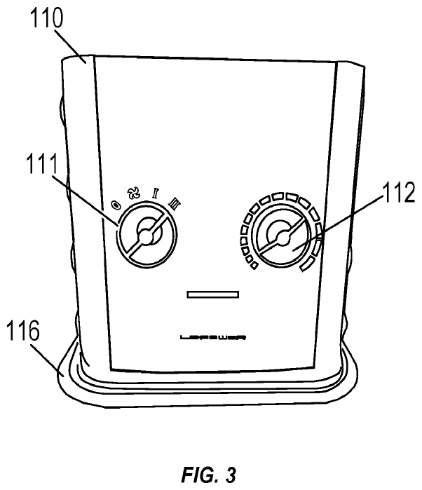

FIGS. 2A-2D show various views of the thawing device of FIG. 1, in accordance with some embodiments. As shown in the front view of FIG. 2A, the air heater unit 110 includes a heat level control 111 and a timer control 112. The heat level control 111 has a knob that can select a fan only setting, a low heat setting and a high heat setting. Each can be selected according to the type and/or temperature of the frozen food FF. The timer control 112 is selectable to a variable range of time, such as between 2 and 10 minutes. Typically, a user would select between 2 and 5 minutes for standard frozen food FF, however, product that has been ultra-frozen may require longer times, such as between 5 to 10 minutes to sufficiently thaw the surface. FIG. 2B shows a top view of the thaw device 100, which shows the air intake 113 in the top of the air heater unit 110. FIG. 2C shows an underside view of the interior of the enclosure cover 120. The heater unit 110 is mounted to the top of the cover 120 so that a heated air outflow 114 is disposed in a top opening of the cover 120. This configuration is advantageous as the heater unit 110 and heated air outflow 114 is fairly isolated from and positioned further from the frozen food product so that the heater unit is not in direct contact with the food product, which avoids the risk of contamination. In some embodiments, the heater unit 110 is readily detachable from the cover so that the cover can be easily removed and washed to avoid cross-contamination when the thawing device is re-used on another unit of frozen food. FIG. 2D shows a tactile textural design 115 on the sides of the heater unit 110, which allows the heater unit to be manually handled by the user when placing the unit atop the container and removing the device after thawing. FIG. 3 shows another view of the front side of the heater unit 110, which shows the bottom side of the heater unit 110 includes a protruding lip 116 that interfaces with the opening in the top of the cover 120.

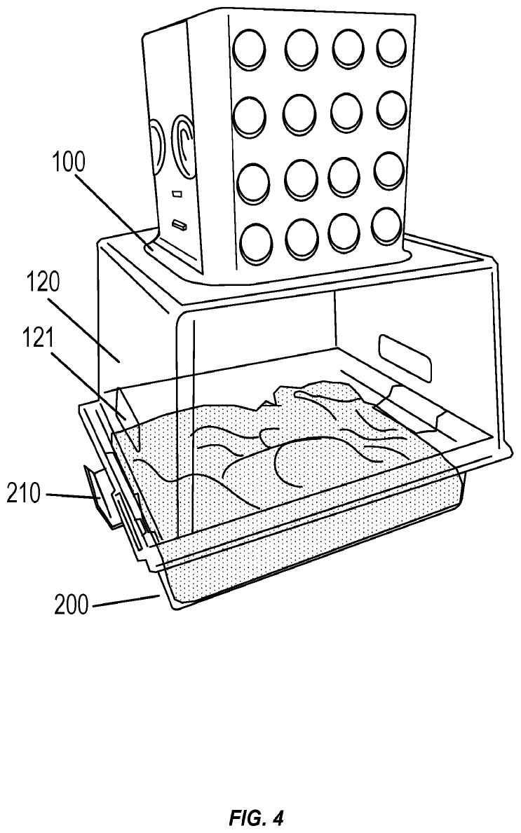

FIGS. 4-9 show steps of sampling frozen food products using a thawing device, in accordance with some embodiments. As shown in FIG. 4, the thawing device has been placed atop a container 200 that contains frozen food, such as frozen beef trim. The operator can optionally secure the cover 120 with a latch mechanism 210 or any suitable coupling mechanism. The cover is sized to correspond to the top opening of the container to create a partial enclosure to direct turbulent airflow of heated air across the surfaces and out through side openings in the cover. After the surface of the frozen food product has been sufficiently thawed for a requisite amount of time, the air heating unit is turned off.

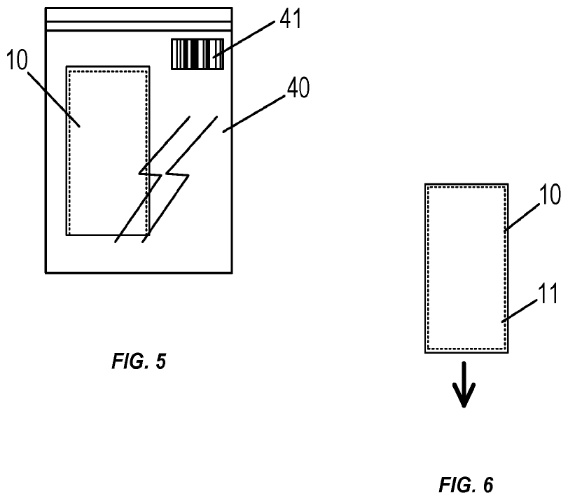

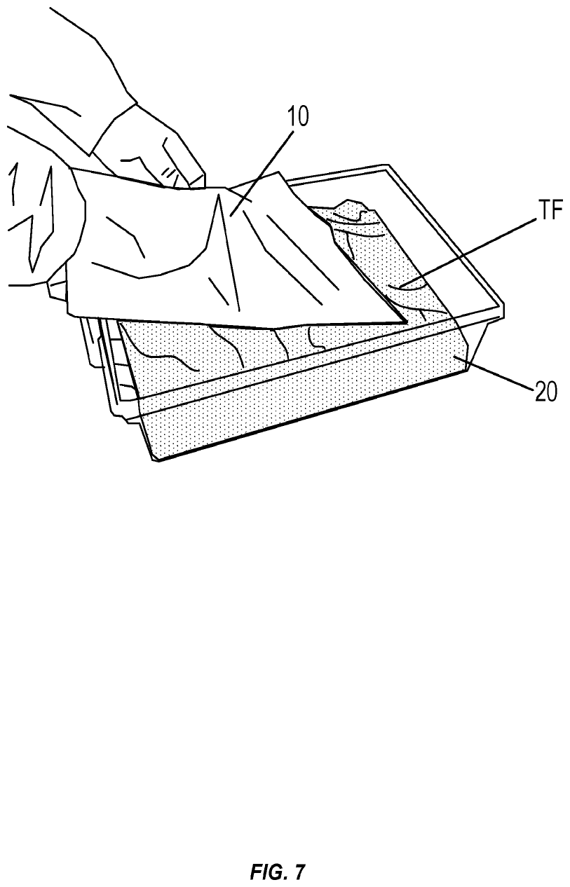

Next, the operator can proceed with obtaining an aggregate sampling from the thawed surface. In order to proceed with a requested sampling of a frozen or solid product, a user obtains an aggregate sampler, which is typically a sterilized sampling sheet or mitt 10 in a sealed bag 40, as shown in FIG. 5. As described previously, the sampling sheet or mitt is fabricated from a suitable microbial sampling medium. The user identifies the sampling sheet or mitt by reading or scanning an identifier 41 (e.g. barcode) on the bag. The sample identifier can be entered or received into an automated or computerized sample tracking system. The identifier can be associated with an identifier of the container or lot number of the frozen food product being sampled. The user then removes the sterilized sampling sheet or mitt 10, as shown in FIG. 6. The mitt 10 can be one or more sampling sheets that include seams 11 to form a pocket for the user's hand. The user then manually contacts the thawed surface of the food product with the sampling sheet or mitt. In the case of a sampling mitt, the user can insert their hand or a tool into a pocket of the mitt before rubbing across the surface of the thawed food TF product, as shown in FIG. 7. The sampling sheet or mitt can be vigorously rubbed across the entire exposed surface for a sufficient duration of time (e.g. between 3 and 60 seconds, 45 seconds), preferably on each side of the sampling sheet to ensure sufficient contact for sampling.

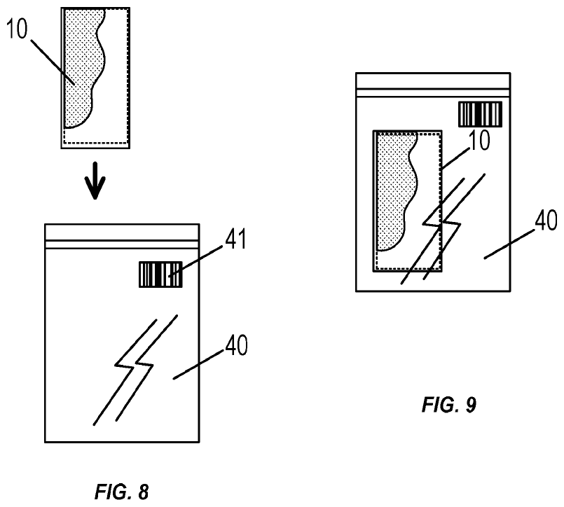

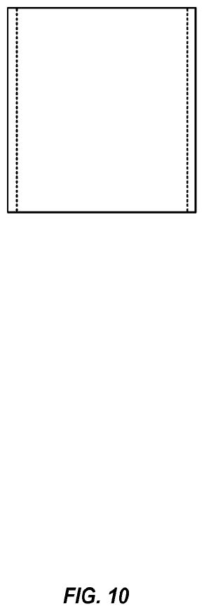

After obtaining the aggregate sampling from the thawed surface of the food product from one or more containers, the user then takes the sampling sheet or mitt and places it back into the sample bag 40, as shown in FIG. 8. In some cases, the user can ascertain that sufficient sample is likely to have been collected by the wetted appearance or staining of the sampling sheet or mitt 10. The user then seals the sampling sheet or mitt 10 within the bag, as shown in FIG. 9, which can then be weighed and compared to a before-sampling weight (either a standard or measured weight) to verify that sufficient weight of liquid sample has been collected. The sample can then be tested according to any appropriate testing method. In some embodiments, the sample can be tested by a procedure that extracts and concentrates the liquid sample for direct testing of the analyte of interest without any enrichment procedure. It is appreciated that any suitable sampling sheet or mitt could be used. In some embodiments, the sampling sheet is a sheet of non-woven polymer material (e.g. MicroTally Sampling Swab), which can be rectangular or square in shape and of a suitable size (e.g. 24×8 inches) to cover a larger sampling surface by manual contact sampling. In other embodiments, the sampling sheet is a folded in half and coupled along opposite lateral edges with seams to form a sampling mitt that a user can cover their hand or a sampling tool. Such a sampling mitt can be about 12×12 inch or smaller, such as 8×12 inches, 8×8 inches, or even smaller such as about 5×5 inches. The smaller sampling mitt are well suited for manually sampling smaller quantities of food product disposed in smaller open containers (e.g. about 12×12″), such as that shown in FIG. 1. An example of a smaller sampling mitt 10′ is shown in FIG. 10. The seams can be formed on opposite lateral sides of a folded sheet such as that the sampling mitt is formed from a single sheet. The seams can be formed from ultrasonic welding or any suitable means. In other embodiments, the sampling mitt can be formed of two equal size sheets that are seamed along three edges.

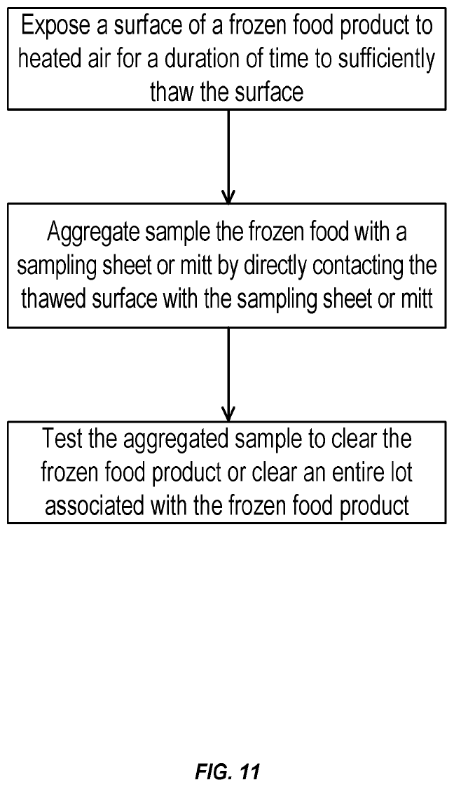

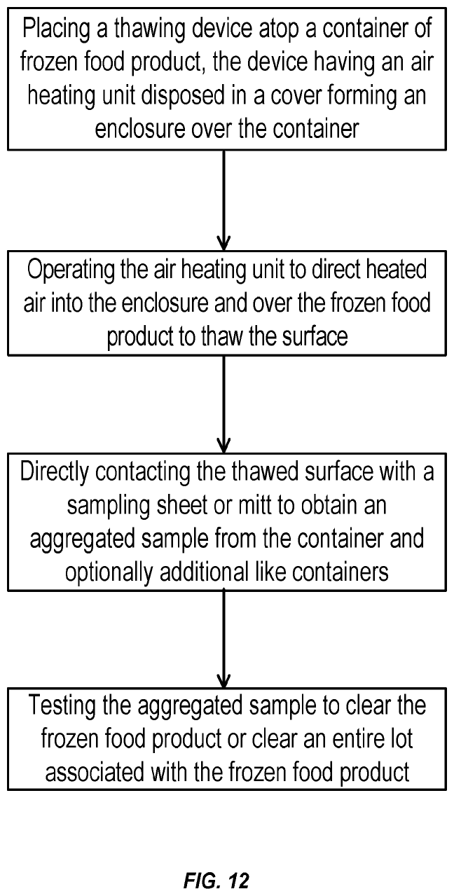

FIGS. 11-12 depict methods of sampling frozen food products using a thawing device, in accordance with some embodiments. FIG. 11 depicts the general concept of the method, which includes: exposing a surface of a frozen food product to heated air for a duration of time to sufficiently thaw a surface; aggregate sampling the top surface with a sampling sheet or mitt by directly contacting the thawed surface with the sampling sheet or mitt; and testing the aggregated sample to clear the frozen food product or clear an entire lot associated with the frozen food product. FIG. 12 depicts additional details of an exemplary method utilizing a thawing device. This method includes steps of: placing a thawing device atop a container of frozen food product, the device having an air heating unit disposed in a cover forming an enclosure over the container; operating the air heating unit to direct heated air into the enclosure and over the frozen food product to thaw the surface; directly contacting the thawed surface with a sampling sheet or mitt to obtain an aggregated sample from the container. Optionally, the method can include repeated this sampling procedure on additional containers. Finally, the method includes testing the aggregated sample to clear the frozen food product or clear an entire lot associated with the frozen food product.

In some embodiments, multiple such thawing devices are provided so that an operator can thaw and sample multiple units in quick succession and improve scalability of this approach. Each sample can be associated with a given lot of food product such that the samples can be used for lot clearance. The lot can be considered the single tray of food product, or can encompass an exceedingly large amount of product (e.g. one or more combo bins). Typically, the operator is expected to open multiple units of product for sampling (e.g. five units is typical). The first unit is heated. This unit is sampled with a suitable sampler (e.g. MicroTally® Mitt or Sheet) while the second unit is heated. This sequential process will continue for all five units. After sampling all units, the sampling medium is bagged for submission for pathogen testing. In this way a single aggregate sampling can be associated with all five units. It is appreciated that the aggregate sample could be associated with more or fewer units, for example, an aggregated sample for each unit, or an aggregate sample for more than five units (e.g. 10, 20, 30, 40, 50 or more units). This approach improves ease of use and scalability in order to sample and clear entire lots of frozen food products.

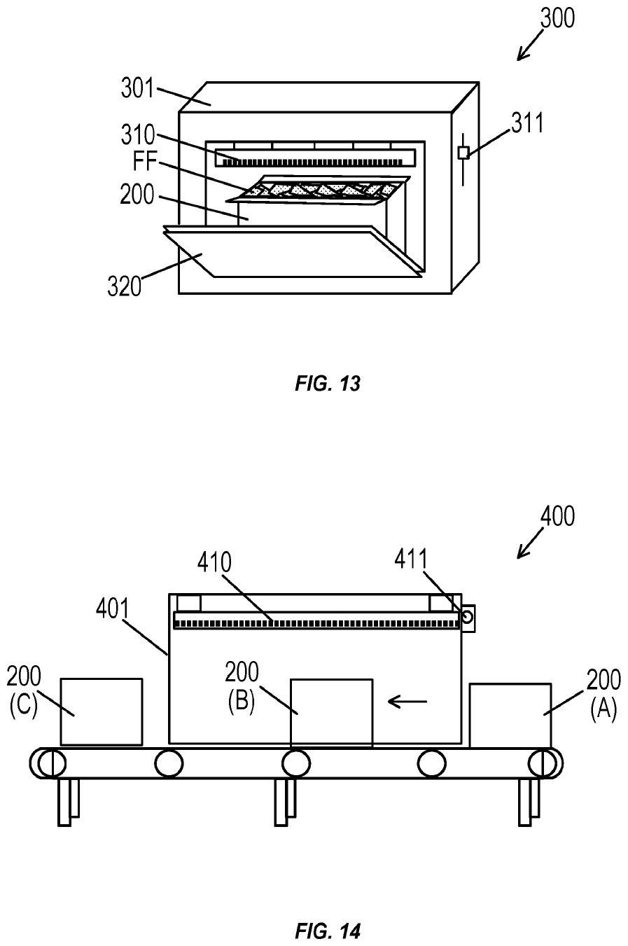

In other embodiments, to facilitate the thawing process, the heating device can be designed as a “melt oven” with adjustable height heating element(s) to accommodate containers of differing sizes, for example, the differing carton sizes listed in Table 1. An example of this embodiment is shown in FIG. 13, which shows a “melt oven” or thawing device 300, which includes an enclosure 301, with an openable door 320 for loading/unloading a container 200 of frozen product, such as frozen food FF. The interior of the enclosure includes one or more heating elements 310, or optionally a heated air unit as described in previous embodiments, the height of which can be adjustable by control 311. It is appreciated that the “melt oven” can further include any of the other features described herein, such as a heat level selector, timer or clock for adjusting, controlling or monitoring application of heat to facilitate thawing. To use the “melt oven,” a user opens the door and places a container 200 of frozen food FF in the interior, adjusts the height of the heating elements to be just above (e.g. within 12 inches, within 6 inches or less) the frozen food FF, then closes the door and allows the heat applied by the heating elements to thaw the surface of the frozen food FF. Once thawed, the door is opened, the container is removed and the thawed surface is sampled, such as by the aggregate sampling technique described above. The melt oven can further include legs to raise the oven further from the floor to facilitate ease of use by sampling personnel.

In still other embodiments, the heating unit mounted over a box conveying system. Such embodiments can optionally include adjustable height element(s) to accommodate containers of differing sizes, for example, the differing carton sizes listed in Table 1. This conveying system can be as simple as roller wheels or as advanced as a motorized belt, such as a conveyor, with a control system. An example of this embodiment is shown in FIG. 14, which shows a conveyance system 400 for transporting a container 200 of frozen product, such as frozen food. A frame 401 suspends one or more heating elements 410, or optionally heated air units as described in previous embodiments, over the conveyor so that the frozen food in the container is heated as it is conveyed, thereby thawing at least the surface. A height adjuster control 401 allows the user to adjust the height of the heating elements according to the height of the container. To use this conveyance heating system, a user places a container 200 filled with frozen product at a proximal region A. The container 200 is conveyed over an intermediate region B during which heat is applied by the heating elements to thaw the surface. Once conveyed to distal region C, the surface of the frozen product in the container is thawed and can be sampled, such as by the aggregate sampling technique described above. Such a system raises the work area and reduces the effort to move boxes which can range up to 60 pounds in weight or more. It is also desirable to increase the stability of the unit even it is not connected to a box conveyance. In some embodiments, adding legs to extend around the box can provide additional stability. The legs could also be adjustable to allow for different carton sizes. This adjustment could include adjustments for height as well as width or depth.

In the foregoing specification, the invention is described with reference to specific embodiments thereof, but those skilled in the art will recognize that the invention is not limited thereto. Various features, embodiments and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. As used herein, the term “about” is +/−10% of the recited value. It is recognized that the terms “comprising,” “including,” and “having,” as used herein, are specifically intended to be read as open-ended terms of art.

What is claimed is:

1. A thawing device to facilitate sampling of frozen food products, the thawing device comprising:
a cover sized to fit over an opening of a container that contains frozen food products such that when the cover is fit over the container, the cover forms an enclosure;
an air heating unit mounted in a top of the cover, the air heating unit having a heated air flow output disposed in an opening in the top of the cover to facilitate heated air flow into the enclosure; and
one or more controls operatively coupled to the air heating unit, wherein the one or more controls are configured to control a heating level and/or a duration of time of operation of the air heating unit so as to thaw a surface of the frozen food for sampling without completely thawing the frozen food product.

2. The thawing device of claim 1, wherein the cover includes one or more openings to allow heated air flow into the enclosure from the air heating unit.

3. The thawing device of any preceding claim, wherein the one or more controls include a heating level control configured to select from multiple heating levels.

4. The thawing device of any preceding claim, wherein the one or more controls further include a timer control configured to select the duration of time of operation of the air heated.

5. The thawing device of any preceding claim, wherein the cover further comprises one or more coupling features to secure the cover atop the container.

6. The thawing device of claim 5, wherein the one or more coupling features comprise one or more latch mechanisms.

7. The thawing device of any preceding claim, wherein the cover is a rectangular or shape.

8. The thawing device of claim 7, wherein the cover is about 12×12 inches about a bottom opening.

9. The thawing device of claim 8, wherein the cover is between 6 and 12 inches in height so as to space the air flow output a suitable distance away from the frozen food provide a more uniform temperature controlled micro-environment in the enclosure and to avoid cross-contamination.

10. A sampling system for sampling frozen foods, the system comprising:
a thawing device as in claim 1;
an aggregate sampler for obtaining an aggregated sample, wherein the aggregate sampler comprises a sampling sheet or mitt configured for obtaining the aggregated sample by direct contact with the thawed surface of multiple products.

11. The sampling system of claim 10, wherein the sampling mitt comprises one or more sheets of absorbent sampling medium comprising a non-woven polypropylene fabric having seams along edges of the one or more sheets to form a pocket for a user's hand to facilitate manual sampling.

12. A method of sampling frozen products, the method comprising:
placing a thawing device atop a container of frozen food products, the device having an air heating unit disposed in a cover forming an enclosure over the container;
operating the air heating unit to direct heated air into the enclosure and over the frozen food product to thaw a surface of the frozen food products; and
sampling the thawed surface of the frozen food product for analytical testing.

13. The method of claim 12, further comprising:
testing the aggregated sample to clear the frozen food product or clear an entire lot associated with the frozen food product.

14. The method of any of claims 12-13, wherein sampling comprises directly contacting the thawed surface with an aggregate sampler to obtain an aggregated sample.

15. The method of claim 14, wherein the aggregate sampler comprises a sampling sheet that is manually rubbed across the thawed surface.

16. The method of claim 14, wherein the aggregate sampler comprises a sampling mitt that is manually rubbed across the thawed surface with a user's hand inside the mitt.

17. The method of any of claims 12-16, further comprising:
selecting, via a control on the heating unit, a heat level based on a type and/or a temperature of the frozen food products.

18. The method of any of claims 12-17, further comprising:
selecting, via a control on the heating unit, a duration of operation of the air heating unit of the frozen food products.

19. The method of any of claims 12-18, further comprising:
after a sufficient duration of time to thaw the surface, ceasing operation of the heating unit.

20. The method of any of claims 12-19, further comprising:
placing the thawing device atop one or more additional containers of frozen food products forming an enclosure over the container;
operating the air heating unit to direct heated air into the enclosure and over the frozen food products to thaw the surface; and
sampling the thawed surface of the frozen food products for analytical testing in order to clear an entire lot associated with the container and one or more additional containers.

21. A thawing device comprising:
a housing defining an enclosure;
a openable door for accessing the enclosure;
one or more heating elements suspended within the enclosure and positioned so as to thaw a top surface of frozen products disposed within a container placed within the enclosure; and
a control for adjusting a height of the one or more heating elements so as to accommodate containers of differing height.

22. A thawing system comprising:
a conveyance system;
a frame disposed on or suspended above the conveyance system;
one or more heating elements supported by the frame and positioned so as to thaw a top surface of frozen products disposed within a container placed on the conveyance system; and
a control for adjusting a height of the one or more heating elements so as to accommodate containers of differing height.
